(19) United States
(12) Patent Application Publication
(10) Pub. No.: US 2024/0366952 A1
(43) Pub. Date: Nov. 7, 2024

(54) Current Mode-Based Programming for Implantable Stimulators

(71) Applicant: Boston Scientific Neuromodulation Corporation, Valencia, CA (US)

(72) Inventors: Bhaskar Soni, Mau (IN); Chirag Shah, Valencia, CA (US); Sultan Salahuddin, Gurgaon (IN); Rachel Fischell, West Hills, CA (US); Peter Yoo, Burbank, CA (US)

(21) Appl. No.: 18/651,034

(22) Filed: Apr. 30, 2024

Related U.S. Application Data
(60) Provisional application No. 63/500,213, filed on May 4, 2023.

(51) Int. Cl. A61N 1/372; A61N 1/05; A61N 1/36

(52) U.S. Cl. CPC A61N 1/37247; A61N 1/0534; A61N 1/36157; A61N 1/36171; A61N 1/36175; A61N 1/36185; A61N 1/37264

(57) ABSTRACT

Methods and systems for assisting a user to program stimulation parameters for an implantable pulse generator (IPG) are described. The IPG may be configured to provide electrical stimulation via a plurality of electrodes. The amount of stimulation current provided by each of the electrodes may be individually controlled. A graphical user interface (GUI) is provided for displaying an indication of the current to be delivered via each the electrodes. In some embodiments, the currents are expressed in terms of a total current to be delivered by the plurality of electrodes, and percentage values of that current to be delivered by each individual electrodes. If a user wishes to modify an amount of current amplitude to be delivered by a particular electrode, the described system is configured to update the total current and the percentage values for each of the electrodes based on the modification.

### CROSS REFERENCE TO RELATED APPLICATIONS

This is a non-provisional of U.S. Provisional Patent Application Ser. No. 63/500,213, filed May 4, 2023, to which priority is claimed, and which is incorporated herein by reference.

### FIELD OF THE INVENTION

This application relates to Implantable Stimulator Devices (ISD), and more specifically to methods and systems for selecting electrodes and stimulation parameters in an ISD such as a Deep Brain Stimulation (DBS) device.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Deep Brain Stimulation (DBS) system, such as that disclosed in U.S. Patent Application Publication 2020/0001091, which is incorporated herein by reference. However, the present invention may find applicability with any implantable neurostimulator device system, including Spinal Cord Stimulation (SCS) systems, Vagus Nerve Stimulation (VNS) system, Sacral Nerve Stimulation (SNS) systems, Peripheral Nerve Stimulation (PNS) systems, and the like.

A DBS system typically includes an Implantable Pulse Generator (IPG) 10 shown in FIG. 1A. The IPG 10 includes a biocompatible device case 12 that holds the circuitry and a battery 14 for providing power for the IPG to function, although the IPG 10 can also lack a battery and can be wirelessly powered by an external source. The IPG 10 is coupled to tissue-stimulating electrodes 16 via one or more electrode leads 18 or 19, which are shown in more details in FIGS. 1B and 1C.

FIG. 1B shows a lead 18 having eight ring-shaped electrodes 16 which are located at different longitudinal positions along a central axis 15. Lead 18 is referred to herein as a “non-directional lead,” because the ring-shaped electrodes span 360 degrees around the axis 15, and thus cannot direct stimulation to different rotational positions around the axis 15.

FIG. 1C shows a lead 19 also having eight electrodes, but not all of the electrodes are ring-shaped. Electrode E8 at the distal end of the lead 19 and electrode E1 at a proximal end of the lead are ring-shaped. Electrodes E2, E3, and E4, by contrast, comprise split-ring electrodes, each of which are located at the same longitudinal position along the axis 15, but each spanning less than 360 degrees around the axis. For example, each of electrodes E2, E3, and E4 may span 90 degrees around the axis 15, with each being separated from the others by gaps of 30 degrees. Electrodes E5, E6, and E7 also comprise split-ring electrodes, but are located at a different longitudinal position. Lead 19 is referred to herein as a “directional lead,” because at least some of the electrodes at a given longitudinal position (e.g., E2, E3, E4) span less than 360 degrees, meaning that those electrodes can direct stimulation to different rotational positions (and hence different brain tissues) around the axis 15. In other designs of a directional lead 19, all electrodes can be split-ring, or there could be different numbers of split-ring electrodes at each longitudinal position (i.e., more or less than three).

Lead wires 20 within the leads are coupled to the electrodes 16 and to proximal contacts 21 insertable into lead connectors 22 fixed in a header 23 on the IPG 10, which header can comprise an epoxy for example. Alternatively, the proximal contacts 21 may connect to lead extensions (not shown) which are in turn inserted into the lead connectors 22. Once inserted, the proximal contacts 21 connect to header contacts 24 within the lead connectors 22, which are in turn coupled by feedthrough pins 25 through a case feedthrough 26 to stimulation circuitry 28 within the case 12, which stimulation circuitry 28 is described below.

In the IPG 10 illustrated in FIG. 1A, there are thirty-two electrodes (E1-E32), split between four percutaneous leads 18 or 19 (18 is shown), and thus the header 23 may include a 2×2 array of eight-electrode lead connectors 22. However, the type and number of leads, and the number of electrodes, in an IPG is application specific and therefore can vary. In another example not shown, a given lead can have 16 sixteen electrodes, and thus this lead would have two sets of proximal contacts 21 to mate with two of the eight-electrode lead connectors 22, as disclosed for example in U.S. Patent Application Publication 2019/0076645. The conductive case 12 can also comprise an electrode (Ec).

In a DBS application, as is useful in the treatment of tremor in Parkinson's disease for example, the IPG 10 is typically implanted under the patient's clavicle (collarbone). Leads 18 or 19 (perhaps as extended by lead extensions, not shown) are tunneled through and under the neck and the scalp, with the electrodes 16 implanted through holes drilled in the skull and positioned for example in the subthalamic nucleus (STN) and the pedunculopontine nucleus (PPN) in each brain hemisphere. The IPG 10 can also be implanted underneath the scalp closer to the location of the electrodes' implantation, as disclosed for example in U.S. Pat. No. 10,576,292. The IPG lead(s) 18 or 19 can be integrated with and permanently connected to the IPG 10 in other solutions.

IPG 10 can include an antenna 27a allowing it to communicate bi-directionally with a number of external devices and systems discussed subsequently. Antenna 27a as shown comprises a conductive coil within the case 12, although the coil antenna 27a can also appear in the header 23. When antenna 27a is configured as a coil, communication with external systems preferably occurs using near-field magnetic induction. IPG 10 may also include a Radio-Frequency (RF) antenna 27b. In FIG. 1A, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Zigbee, WiFi, MICS, and the like. If the IPG 10 lacks a battery 14, an additional coil can be present to receive wireless power from an external source.

Stimulation in IPG 10 is typically provided by pulses each of which may include a number of phases such as 30a and 30b, as shown in the example of FIGS. 2A and 2B. In the example shown, such stimulation is monopolar, meaning that a current is provided between at least one selected lead-based electrode (e.g., E1) and the case electrode Ec 12. Stimulation could be bipolar, in which a current is provided between at least two lead-based electrodes. Stimulation parameters typically include amplitude (current I, although a voltage amplitude V can also be used); frequency (F); pulse width (PW) of the pulses or of its individual phases such as 30a and 30b; the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2A, electrode E1 has been selected as a cathode (during its first phase 30a), and thus provides pulses which sink a negative current of amplitude −I from the tissue. The case electrode Ec has been selected as an anode (again during first phase 30a), and thus provides pulses which source a corresponding positive current of amplitude +I to the tissue. Note that at any time the current sunk from the tissue (e.g., −I at E1 during phase 30a) equals the current sourced to the tissue (e.g., +I at Ec during phase 30a). The polarity of the currents at these electrodes can be changed: for example, during first phase 30a, Ec can be selected as a cathode, and E1 can be selected as an anode, etc.

IPG 10 as mentioned includes stimulation circuitry 28 to form prescribed stimulation at a patient's tissue. FIG. 3 shows an example of stimulation circuitry 28, which includes one or more current sources 40; and one or more current sinks 42ᵢ. The sources and sinks 40; and 42; can comprise Digital-to-Analog converters (DACs), and may be referred to as PDACs 40; and NDACs 42; in accordance with the Positive (sourced, anodic) and Negative (sunk, cathodic) currents they respectively issue. In the example shown, a NDAC/PDAC 40ᵢ/42ᵢ pair is dedicated (hardwired) to a particular electrode node ei 39. Each electrode node ei 39 is connected to an electrode Ei 16 via a DC-blocking capacitor Ci 38, for the reasons explained below. PDACs 40; and NDACs 42; can also comprise voltage sources. The stimulation circuitry 28 may also be referred to herein as DAC circuitry.

Proper control of the PDACs 40; and NDACs 42; allows any of the electrodes 16 and the case electrode Ec 12 to act as anodes or cathodes to create a current (such as the pulses described earlier) through a patient's tissue, Z, hopefully with good therapeutic effect. In the example shown, and consistent with the first pulse phase 30a of FIG. 2A, electrode E1 has been selected as a cathode electrode to sink current from the tissue Z and case electrode Ec has been selected as an anode electrode to source current to the tissue Z. Thus PDAC 40c and NDAC 421 are activated and digitally programmed to produce the desired current, I, with the correct timing (e.g., in accordance with the prescribed frequency F and pulse width PW). Power for the stimulation circuitry 28 is provided by a compliance voltage VH, as described in further detail in U.S. Patent Application Publication 2013/0289665. Other stimulation circuitries 28 can also be used in the IPG 10. In an example not shown, a switching matrix can intervene between the one or more PDACs 40; and the electrode nodes ci 39, and between the one or more NDACs 42; and the electrode nodes. Switching matrices allows one or more of the PDACs or one or more of the NDACs to be connected to one or more electrode nodes at a given time. Various examples of stimulation circuitries can be found in U.S. Pat. Nos. 6,181,969, 8,606,362, 8,620,436, U.S. Patent Application Publications 2018/0071520 and 2019/0083796.

Much of the stimulation circuitry 28 of FIG. 3, including the PDACs 40ᵢ and NDACs 42ᵢ, the switch matrices (if present), and the electrode nodes ci 39 can be integrated on one or more Application Specific Integrated Circuits (ASICs), as described in U.S. Patent Application Publications 2012/0095529, 2012/0092031, and 2012/0095519. As explained in these references, ASIC(s) may also contain other circuitry useful in the IPG 10, such as telemetry circuitry (for interfacing off chip with telemetry antennas 27a and/or 27b), circuitry for generating the compliance voltage VH, various measurement circuits, etc.

Also shown in FIG. 3 are DC-blocking capacitors Ci 38 placed in series in the electrode current paths between each of the electrode nodes ei 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861.

Referring again to FIG. 2A, the stimulation pulses as shown are biphasic, with each pulse comprising a first phase 30a followed thereafter by a second phase 30b of opposite polarity. Biphasic pulses are useful to actively recover any charge that might be stored on capacitive elements in the electrode current paths, such as on the DC-blocking capacitors 38, as is well known. FIG. 3 also shows that stimulation circuitry 28 can include passive recovery switches 41ᵢ, which are described further in U.S. Patent Application Publications 2018/0071527 and 2018/0140831. Passive recovery switches 41; may be closed to passively recover any charge remaining on the DC-blocking capacitors Ci 38 after issuance of the second pulse phase 30b—i.e., to recover charge without actively driving a current using the DAC circuitry, as shown during duration 30c. Alternatively, passive charge recovery can be used during the second pulse phase 30b after the actively driven first pulse phase 30a, although this isn't shown in FIG. 2A. Again, passive charge recovery is well known and not further described.

FIG. 4 shows various external systems 60, 70, and 80 that can wirelessly communicate data with the IPG 10. Such systems can be used to wirelessly transmit a stimulation program to the IPG 10—that is, to program its stimulation circuitry 28 to produce stimulation with desired amplitudes and timings as described earlier. Such systems may also be used to adjust one or more stimulation parameters of a stimulation program that the IPG 10 is currently executing, and/or to wirelessly receive information from the IPG 10, such as various status information and measurements, etc.

External controller 60 can be as described in U.S. Patent Application Publication 2015/0080982 for example, and may comprise a portable, hand-held controller dedicated to work with the IPG 10. External controller 60 may also comprise a general-purpose mobile electronics device such as a mobile phone which has been programmed with a Medical Device Application (MDA) allowing it to work as a wireless controller for the IPG 10, as described in U.S. Patent Application Publication 2015/0231402. External controller 60 includes a display 61 and a means for entering commands, such as buttons 62 or selectable graphical icons provided on the display 61. The external controller 60's user interface enables a patient to adjust stimulation parameters, although it may have limited functionality when compared to systems 70 and 80, described shortly. The external controller 60 can have one or more antennas capable of communicating with a compatible antenna in the IPG 10, such as a near-field magnetic-induction coil antenna 64a and/or a far-field RF antenna 64b.

Clinician programmer 70 is described further in U.S. Patent Application Publication 2015/0360038, and can comprise a computing device such as a desktop, laptop, or notebook computer, a tablet, a mobile smart phone, a Personal Data Assistant (PDA)-type mobile computing device, etc. In FIG. 4, the computing device is shown as a laptop computer that includes typical computer user interface means such as a display 71, buttons 72, as well as other user-interface devices such as a mouse, a keyboard, speakers, a stylus, a printer, etc., not all of which are shown for convenience. Also shown in FIG. 4 are accessory devices for the clinician programmer 70 that are usually specific to its operation as a stimulation controller. A communication “wand” 76 coupleable to suitable ports on the computing device can include an IPG-compliant antenna such as a coil antenna 74a or an RF antenna 74b. The computing device itself may also include one or more RF antenna 74b. The clinician programmer 70 can also communicate with other devices and networks, such as the Internet, either wirelessly or via a wired link provided at an Ethernet or network port.

External system 80 comprises another means of communicating with and controlling the IPG 10 via a network 85 which can include the Internet. The network 85 can include a server 86 programmed with IPG communication and control functionality, and may include other communication networks or links such as WiFi, cellular or land-line phone links, etc. The network 85 ultimately connects to an intermediary device 82 having antennas suitable for communication with the IPG's antenna, such as a near-field magnetic-induction coil antenna 84a and/or a far-field RF antenna 84b. Intermediary device 82 may be located generally proximate to the IPG 10. Network 85 can be accessed by any user terminal 87, which typically comprises a computer device associated with a display 88. External system 80 allows a remote user at terminal 87 to communicate with and control the IPG 10 via the intermediary device 82.

FIG. 4 also shows circuitry 90 involved in any of external systems 60, 70, or 80. Such circuitry can include control circuitry 92, which can comprise any number of devices such as one or more microprocessors, microcomputers, FPGAs, DSPs, other digital logic structures, etc., which are capable of executing programs in a computing device. Such control circuitry 92 may contain or coupled with memory 94 which can store external system software 96 for controlling and communicating with the IPG 10, and for rendering a Graphical User Interface (GUI) 99 on a display (61, 71, 88) associated with the external system. In external system 80, the external system software 96 would likely reside in the server 86, while the control circuitry 92 could be present in either or both the server 86 or the terminal 87.

### SUMMARY

Disclosed herein is a system for programming an implantable pulse generator (IPG), wherein the IPG is configurable to connect to a plurality of electrodes, the system comprising: control circuitry configured to: provide a graphical user interface (GUI) displaying an indication of an initial total current to be delivered by the plurality of electrodes and, for each of the electrodes, an initial percentage value indicating an initial percentage of the initial total current assigned to that electrode, wherein each initial percentage value defines an initial amplitude of current assigned to that electrode; receive, via the GUI, an instruction to adjust the initial amplitude assigned to a selected one of the electrodes to an adjusted amplitude while maintaining the initial amplitudes for each of the non-selected electrodes; calculate an adjusted total current and adjusted percentage values for each of the plurality of electrodes in accordance with the instruction, and use the adjusted total current and the adjusted percentage values to program the IPG. According to some embodiments, the initial percentage values are configured to provide stimulation at a virtual pole at a position that is not a position of any of the electrodes. According to some embodiments, the GUI displays graphical representations of each of the plurality of electrodes such that each of the graphical representations are selectable by a user. According to some embodiments, the GUI displays GUI elements for adding and/or subtracting amplitude of current assigned to the selected electrode. According to some embodiments, calculating an adjusted total current adding the initial amplitudes of the non-selected electrodes and the adjusted amplitude of the selected electrode. According to some embodiments, calculating the adjusted percentage values for each of the plurality of electrodes comprises: calculating an adjusted percentage value for the selected electrode by dividing the adjusted current for the selected electrode by the adjusted total current, and calculating adjusted percentage values for each of the non-selected electrodes by dividing the initial amplitude for that that non-selected electrode by the adjusted total current. According to some embodiments, using the adjusted total current and the adjusted percentage values to program the IPG comprises: determining if the IPG is capable of producing the adjusted total current and the adjusted percentage values, if the IPG is capable of producing the adjusted total current and the adjusted percentage values, transmitting the adjusted total current and the adjusted percentage values to the IPG, and if the IPG is not capable of producing the adjusted total current and the adjusted percentage values, calculating a modified adjusted total current and modified adjusted percentage values. According to some embodiments, determining if the IPG is capable of producing the adjusted total current and the adjusted percentage values comprises determining if the adjusted total current and the adjusted percentage values are within resolution capabilities of digital-to-analog (DAC) circuitry of the IPG. According to some embodiments, the modified adjusted total current and modified adjusted percentage values are calculated to minimize a deviation between the initial amplitude of current assigned to at least one of the non-elected electrodes and an amplitude of current defined for that non-elected electrode by the modified adjusted total current and modified adjusted percentage values. According to some embodiments, the GUI is configured to display an indication of the deviation between the initial amplitude of current assigned to the at least one of the non-elected electrodes and the amplitude of current defined for that non-elected electrode by the modified adjusted total current and modified adjusted percentage values. According to some embodiments, the control circuitry is configured to compare the deviation to a predetermined threshold value for the deviation and issue a warning if the deviation exceeds the threshold value. According to some embodiments, the GUI is configured to allow a user to set the predetermined threshold value for any of the plurality of electrodes. According to some embodiments, the GUI is configured to display the amplitude of current assigned to any of the plurality of electrodes.

Also disclosed herein is a system for programming an implantable pulse generator (IPG), wherein the IPG is configurable to connect to a plurality of electrodes, the system comprising: control circuitry configured to: receive an indication of an amplitude of current to be assigned to a selected electrode, use the amplitude of current to be assigned to the selected electrode to calculate a total current to be delivered by the plurality of electrodes and, for each of the electrodes, a percentage value indicating a percentage of the total current assigned to that electrode, and transmit an indication of the total current and the percentage values to the IPG. According to some embodiments, calculating the total current comprises adding the amplitude of current assigned to the selected electrode to amplitudes of currents assigned to non-selected electrodes. According to some embodiments, calculating the percentage values for each of the plurality of electrodes comprises: calculating a percentage value for the selected electrode by dividing the current for the selected electrode by the adjusted total current, and calculating percentage values for each of the non-selected electrodes by dividing the amplitude for that that non-selected electrode by the total current. According to some embodiments, the control circuitry is further configured to use the total current and the percentage values to program the IPG. According to some embodiments, the total current and the percentage values to program the IPG comprises: determining if the IPG is capable of producing the total current and the percentage values, if the IPG is capable of producing the total current and the percentage values, transmitting the total current and the percentage values to the IPG, and if the IPG is not capable of producing the total current and the percentage values, calculating a modified total current and modified percentage values. According to some embodiments, determining if the IPG is capable of producing the total current and the percentage values comprises determining if the total current and the percentage values are within resolution capabilities of digital-to-analog (DAC) circuitry of the IPG. According to some embodiments, the modified total current and modified percentage values are calculated to minimize a deviation between the initial amplitude of current assigned to at least one of the non-elected electrodes and an amplitude of current defined for that non-elected electrode by the modified total current and modified percentage values.

Also disclosed herein is non-transitory computer-readable media comprising instructions, which, when executed by a computer, configure the computer to perform any of the steps described above.

### BRIEF DESCRIPTION OF THE DRAWINGS

FIG. 1A shows an Implantable Pulse Generator (IPG), in accordance with the prior art.

FIG. 1B shows a percutaneous lead having ring electrodes, and FIG. 1C shows a percutaneous lead having split ring electrodes, in accordance with the prior art.

FIGS. 2A and 2B show an example of stimulation pulses (waveforms) producible by the IPG, in accordance with the prior art.

FIG. 3 shows an example of stimulation circuitry useable in the IPG, in accordance with the prior art.

FIG. 4 shows various external systems capable of communicating with and programming stimulation in an IPG, in accordance with the prior art.

FIG. 5A shows a Graphical User Interface (GUI) operable on an external system such as a clinician programmer, which is capable of programming a stimulation program for the IPG.

FIG. 5B shows waveforms produced at the electrodes through use of the GUI of FIG. 5A.

FIG. 6 shows changing an amount of amplitude current at an electrode using current mode (i.e., mA mode) programming.

FIG. 7 shows an embodiment for adjusting an amount of amplitude current at an electrode using current mode (i.e., mA mode) programming.

FIG. 8 shows tracking of deviations in current at electrodes.

FIG. 9 shows an embodiment of an algorithm for programming electrode current amplitudes using current (mA) mode programming.

### DETAILED DESCRIPTION

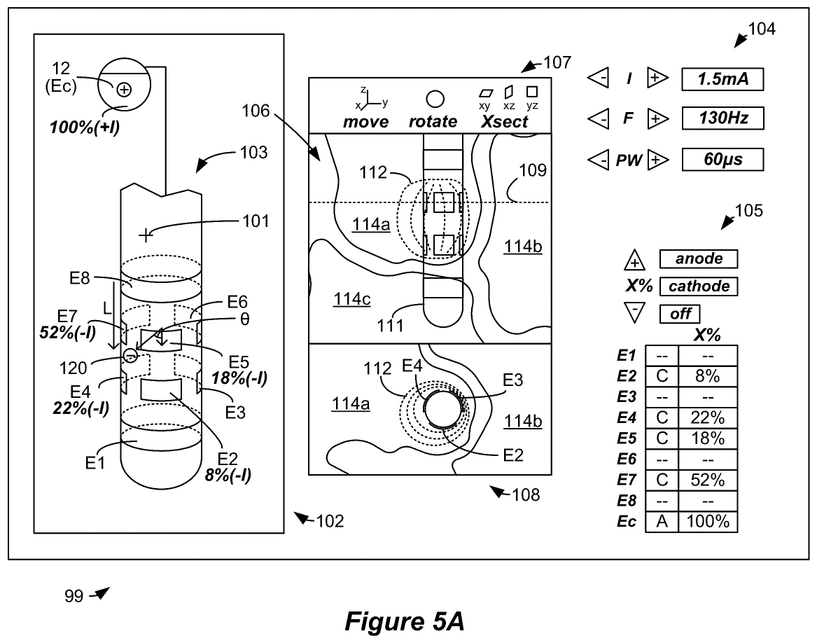

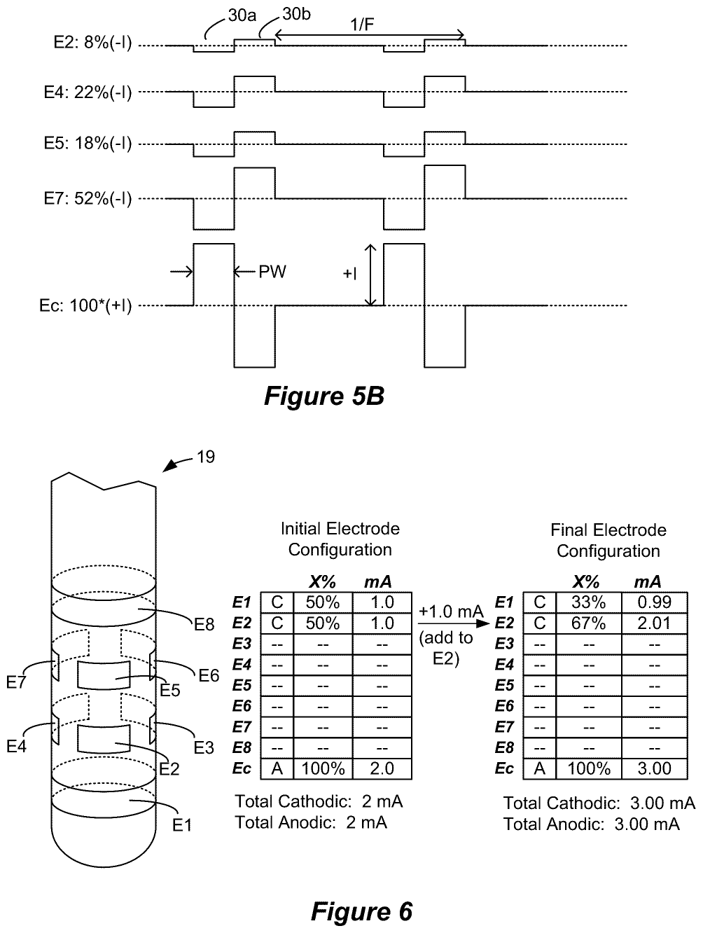

FIG. 5A shows an example of GUI 99 rendered on the display of an external system, such as the clinician programmer 70 mentioned earlier. GUI 99 is particularly useful in an DBS context because it provides a clinician with a visual indication of how stimulation selected for a patient will interact with the brain tissue in which the electrodes are implanted. GUI 99 can be used during surgical implantation of the leads 18 or 19 and its IPG 10, but can also be used after implantation to assist in selecting a therapeutically useful stimulation program for the patient. The GUI 99 can be controlled by a cursor 101 that the user can move using a mouse connected to the clinician programmer 70 for example.

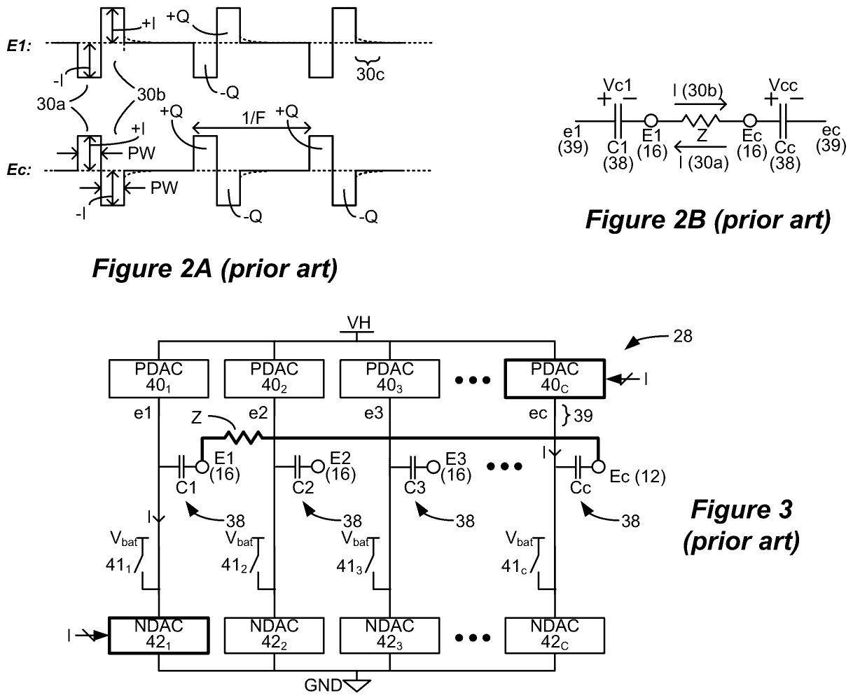

The GUI 99 may include a waveform interface 104 where various aspects of the stimulation can be selected or adjusted. For example, waveform interface 104 allows a user to select an amplitude (e.g., a current I), a frequency (F), and a pulse width (PW) of the stimulation pulses. Waveform interface 104 can be significantly more complicated, particularly if the IPG 10 supports the provision of stimulation that is more complicated than a repeating sequence of pulses. Waveform interface 104 may also include inputs to allow a user to select whether stimulation will be provided using biphasic (FIG. 2A) or monophasic pulses, or in bursts of pulses, and to select whether passive charge recovery will be used, although again these details aren't shown for simplicity.

The GUI 99 may also include an electrode configuration interface 105 which allows the user to select a particular electrode configuration specifying which electrodes should be active to provide the stimulation, and with which polarities and relative magnitudes. In this example, the electrode configuration interface 105 allows the user to select whether an electrode should comprise an anode (A) or cathode (C) or be off, and allows the amount of the total anodic or cathodic current +I or −I (specified in the waveform interface 104) that each selected electrode will receive to be specified in terms of a percentage, X. For example, in FIG. 5A, the case electrode 12 Ec is specified to be an anode that receives X=100% of the current I as an anodic current +I (e.g., during first pulse phase 30a if biphasic pulses are used; see FIG. 2A). The corresponding cathodic current −I is split between cathodes electrodes E2 (8% or 0.08*−I), E4 (22% or 0.22*−I), E5 (18% or 0.18*−I), and E7 (52% or 0.52*−I) (again during first pulse phase 30a). The waveforms resulting at the electrodes from this electrode configuration are shown in FIG. 5B. Note that two or more electrodes can be chosen to act as anodes or cathodes at a given time, allowing the electric field in the tissue to be shaped, as explained further below. Once the waveform parameters (104) and electrode configuration parameters (105) are determined, they can be sent from the clinician programmer 70 to the IPG 10, so that the IPG's stimulation circuitry 28 (FIG. 3) can be programmed (the various NDACs and PDACs) to produce the desired currents at the selected electrodes with the proper timing. For example, PDAC 40c would be programmed to produce +100%*+I, and NDAC 424 would be programmed to produce 22%*−I, etc. Together, the various waveform parameters and electrode configuration parameter comprise stimulation parameters, which together comprise a stimulation program.

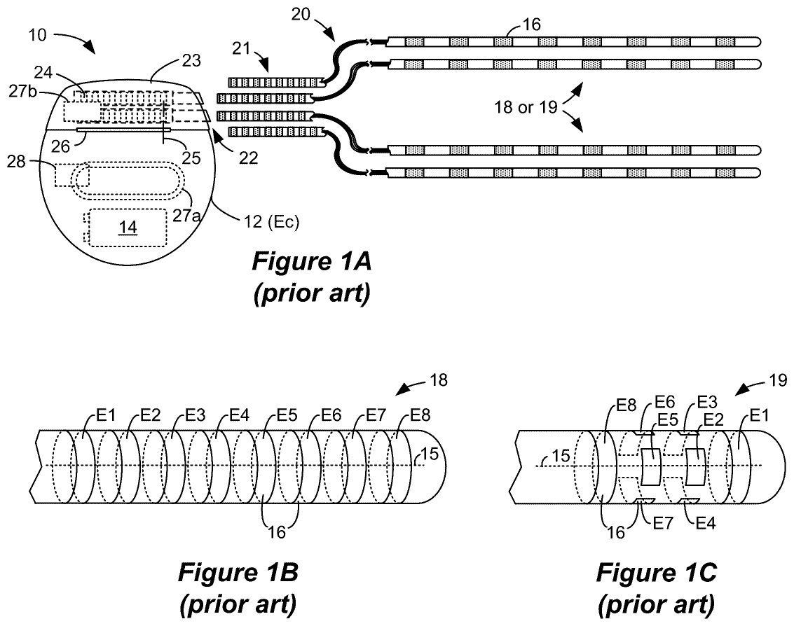

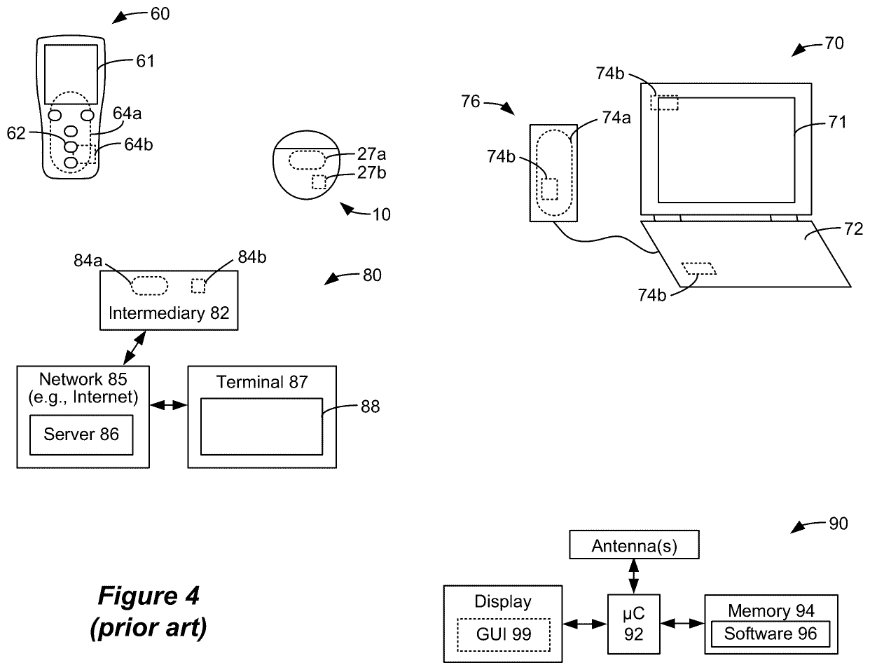

Use of these electrodes to provide cathodic stimulation sets a particular position for a cathodic pole 120 in three-dimensional space, as shown in FIG. 5A. The position of this cathode pole 120 can be quantified at a particular longitudinal position L along the lead (e.g., relative to a point on the lead such as the longitudinal position of electrode E1), and at a particular rotational angle θ (e.g., relative to a particular angle on the lead such as relative to the center of electrode E2). (Note that rotation angle θ is only relevant when a directional lead such as 19 (FIG. 1C) is used). This position is shown in a leads interface 102 of the GUI 99. Notice that the position of the pole 120 (L,θ) may be virtual; that is, the position may not necessarily occur at the physical position of any of the electrodes 16 in the electrode array, as explained further later. The leads interface 102 preferably also includes an image 103 of the lead being used for the patient. Although not shown, the leads interface 102 can include a selection to access a library of relevant representations 103 of the types of leads (e.g., 18 or 19) that may be implanted in different patients, which may be stored with the relevant software (e.g., 96, FIG. 4). The cursor 101 can be used to select an illustrated electrode 16 (e.g., E1-E8, or the case electrode Ec), or a pole such as cathode pole 120. Pole 120 could also be anodic, or there could be more than one pole if multipolar stimulation is used, but this isn't shown.

An electrode configuration algorithm (not shown), operating as part of external device's software 96, can determine a position of the cathode pole 120 in three-dimensional space from a given electrode configuration, and can also conversely determine an electrode configuration from a given position of the pole 120. For example, the user can place the position of the pole 120 using the cursor 101. The electrode configuration algorithm can then be used to compute an electrode configuration that best places the pole 120 in this position. Note that cathode pole 120 is positioned closest to electrode E7, but is also generally proximate to electrodes E5, E4, and E2. The electrode configuration algorithm may thus calculate that electrode E7 should receive the largest share of cathodic current (52%*−I), while E5, E4, and E2 which are farther away from the pole 120 receive lesser percentages, as shown in the stimulation parameters interface 104. By involving more than one electrode, cathode pole 120 is formed as a virtual pole not as the position of any of the physical electrodes. Again, the electrode configuration algorithm can also operate in reverse: from a given electrode configuration, the position of the pole 120 can be determined. The electrode configuration algorithm is described further in U.S. Patent Application Publication 2019/0175915, which is incorporated herein by reference. Ultimately, the external system upon which the GUI is operating provides instructions to the IPG that define (1) the total amount of current (cathodic and/or anodic) that the electrode lead will provide, and (2) how that current will be fractionated among the various electrodes on the lead (i.e., the percentage of the total current that each electrode will deliver).

GUI 99 can further include a visualization interface 106 that allows a user to view a stimulation field image 112 formed on a lead given the selected stimulation parameters and electrode configuration. The stimulation field image 112 is formed by field modelling in the clinician programmer 70, as discussed further in the '091 Publication. The visualization interface 106 preferably, but not necessarily, further includes tissue imaging information 114. This tissue imaging information 114 is presented in FIG. 5A as three different tissue structures 114a, 114b and 114c in FIG. 6 for the patient in question, which tissue structures may comprise different areas of the brain for example. Such tissue imaging information may come from a Magnetic Resonance Image (MRI) or Computed Tomography (CT) image of the patient, may come from a generic library of images, and may include user defined regions. The GUI 99 can overlay the lead image 111 and the stimulation field image 112 with the tissue imaging information 114 in the visualization interface 106 so that the position of the stimulation field 112 relative to the various tissue structures 114i can be visualized. The various images shown in the visualization interface 106 (i.e., the lead image 111, the stimulation field image 112, and the tissue structures 114i) can be three-dimensional in nature, and hence may be rendered to allow such three-dimensionality to be better appreciated by the user, such as by shading or coloring the images, etc. A view adjustment interface 107 may allow the user to move or rotate the images, using cursor 101 for example, as explained in the '091 Publication. In FIG. 5A, a cross-section interface 108 allows the various images to be seen in a particular two-dimensional cross section, and in this example a cross section 109 is shown taken perpendicularly to the lead image 111 and through split-ring electrodes E2, E3, and E4. Interfaces 106 and 108 may also show the cathode pole 120 in a proper position, but this isn't shown.

The GUI 99 of FIG. 5A is particularly useful because it allows the electric field as reflected in stimulation field image 112 (or the pole 120) to be seen relative to surrounding tissue structures 114i. This allows the user to adjust the stimulation parameters to recruit, or avoid recruiting, particular tissue structures 114i. Assume for example that it is desirable for a given patient to stimulate tissue structure 114a, but to not stimulate tissue structures 114b or 114c. This may be because tissue structure 114a is causing undesired patient symptoms (e.g., tremor) that stimulation can alleviate, while stimulation of tissue structures 114b and 114c will cause undesired side effects. The clinician can then use GUI 99 to adjust stimulation (e.g., to adjust the stimulation parameters or the electrode configuration) to move the stimulation field 112 (e.g., the cathode pole 120) to a proper position (L, θ). In the example shown, and as best seen in the cross-section interface 108, higher cathodic currents are provided at split-ring electrodes E7 (0.52*−I) and E5 (0.18*−I) because these electrodes are generally facing towards tissue structure 114a which should be stimulated. By contrast, split-ring electrode E6 carries no cathodic current because it generally faces towards tissue structure 114b where stimulation is ideally avoided. The result is a stimulation field 112 that is more predominant in tissue structure 114a and less predominant in tissue structure 114b, as shown in the visualization interface 106.

Especially in a DBS application, it is important that correct stimulation parameters be determined for a given patient. Improper stimulation parameters may not yield effective relief of a patient's symptoms, or may cause unwanted side effects. To determine proper stimulation, a clinician typically uses GUI 99 to try different combinations of stimulation parameters. This may occur, at least in part, during a DBS patient's surgery when the leads are being implanted. Such intra-operative determination of stimulation parameters can be useful to determine a general efficacy of DBS therapy. However, finalizing stimulation parameters that are appropriate for a given DBS patient typically occurs after surgery after the patient has had a chance to heal, and after the position of the leads stabilize in the patient. Thus, at such time, the patient will typically present to the clinician's office to determine (or further refine) optimal stimulation parameters during a programming session.

Gauging the effectiveness of a given set of stimulation parameters typically involves programming the IPG 10 with that set, and then reviewing the therapeutic effectiveness and side effects that result. Therapeutic effectiveness and side effects are often assessed by one or more different scores(S) for one or more different clinical responses, which are entered into the GUI 99 of the clinician programmer 70 where they are stored with the stimulation parameters set being assessed. Such scores can be subjective in nature, based on patient or clinician observations. For example, bradykinesia (slowness of movement), rigidity, tremor, or other symptoms or side effects, can be scored by the patient, or by the clinician upon observing or questioning the patient. Such scores in one example can range from 0 (best) to 4 (worst).

Scores can also be objective in nature based on measurements taken regarding a patient's symptoms or side effects. For example, a Parkinson's patient may be fitted with a wearable sensor that measures tremors, such as by measuring the frequency and amplitude of such tremors. A wearable sensor may communicate such metrics back to the GUI 99, and if necessary, converted to a score. U.S. Patent Application Publication 2021/0196956, which is incorporated herein by reference in its entirety, discusses determining which symptoms and/or side effects are most sensible to score for a given patient when the stimulation parameters are optimized.

Aspects of this disclosure relate to methods and systems for allowing a clinician to more easily program stimulation currents that will be applied at the lead's electrodes. In particular, the described methods and systems allow a clinician to easily add or subtract current from one or more selected electrodes without significantly changing the current programmed at other electrodes. Note that the examples described herein mainly relate to applying cathodic current at the electrodes situated on the electrode lead and using the case as a counter electrode to apply anodic current. It will be appreciated that lead electrodes may apply one or more of anodic or cathodic current and the case may or may not be used as a counter electrode. In many instances herein, the counter electrode(s) will be ignored/omitted, for clarity.

Consider a situation as illustrated in FIG. 6. Assume an initial condition wherein a total cathodic current of 2 mA is applied using an electrode lead 19 and that current is equally divided between E1 and E2. The case electrode serves as a counter electrode and sinks 2 mA of anodic current. Now assume that the clinician wants to add an additional 1 mA of cathodic current to E2. But the clinician does not want the current on E1 to change, for example, because the change may have an adverse effect on the patient. Recall from the above discussion the clinician may interact with a GUI (e.g., GUI 99, FIG. 5A) presented on a clinician programmer 70 (FIG. 4) to make changes to the electrode configuration. Also recall that, according to some embodiments, the GUI provides instructions to the IPG that define (1) the total amount of current (cathodic and/or anodic) that the electrode lead will provide, and (2) how that current will be fractionated among the various electrodes on the lead (i.e., the percentage of the total current that each electrode will deliver). In such an embodiment, the clinician would need to calculate the total amount of current to add to the electrode lead and calculate the new fractionation of that current among each of the electrodes that would result in the desired change. In the illustrated example, the new total cathodic current would be 3.0 mA, with 33.33 . . . % of the current provided at E1 and 66.66 . . . % of the current provided at E2. The clinician would then need to provide the new total current value and the new calculated percentage values to the IPG via the GUI. This recalculation of the appropriate total current and the appropriate percentages of the current to apply to each electrode is time consuming. The calculations may become quite cumbersome as the number of changes and/or the number of electrodes to be changed increases. Moreover, the architecture of the IPG, such as the IPG's DAC circuitry (e.g., 40/42, FIG. 3) may present limitations as to the percentage values that may be used, as explained in more detail below. For now, note that such architecture limitations are a reason that the current on E1 changed from 1.0 mA to 0.99 mA and the new current on E2 is 2.01 mA (instead of the even 2 mA that the clinician specified).

Embodiments of the instant disclosure allow a clinician to add/subtract current from a given electrode without (or minimally) affecting other electrodes. More broadly, embodiments of the disclosure allow electrode currents to be programmed using either a percentage (fraction) basis or a current (i.e., mA) basis.

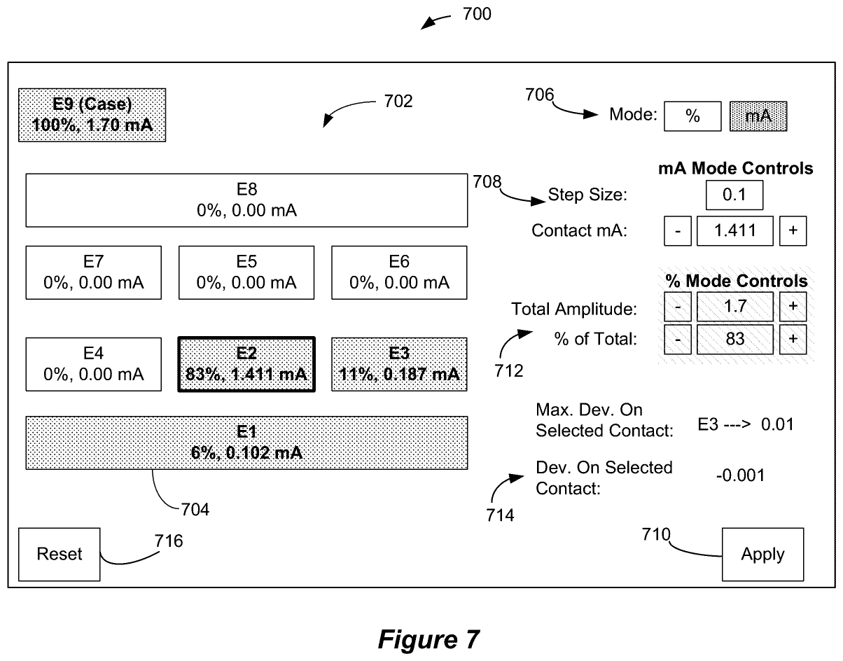

FIG. 7 illustrates an example of a GUI 700 according to some embodiments of the application. The features of the GUI 700 may be included as additional elements of the GUI 99 (FIG. 5A) or otherwise presented on a clinician programmer 70 (FIG. 4), for example. The GUI includes a representation 702 of each of the electrodes on the lead (and the case/counter electrode). Note that the representation 702 illustrated in FIG. 7 is a two-dimensional array of GUI electrode elements 704, such as would be obtained if the electrodes of a directional lead (e.g., electrode lead 19, FIG. 6) were flattened out. According to other embodiments, the electrode lead and its electrodes could be represented in three dimensions (like the representation 103 of FIG. 5A). The GUI electrode elements for each electrode may comprise an indication of the amount and/or percentage of total current assigned to be delivered by the electrode. According to some embodiments, electrodes that are active (i.e., they are assigned to deliver current) may be highlighted or colored to show that they are active. The user may select particular electrodes to adjust (i.e., by a mouse click, touch screen, or by entering text). According to some embodiments, the selected electrode element may be further highlighted to indicate that is selected for adjustment. In the illustration, E2 is bolded, indicating that it is the electrode element being adjusted.

The GUI may include mode selection elements 706, whereby the user can choose to adjust the electrode currents using either percentage (fractionation) mode or current mode. By selecting current mode (i.e., mA mode), the user can add or subtract current from the selected electrode element. The GUI may include current mode control elements 708, whereby the user can select a step size and an amount of current to add or subtract from the selected electrode. As the user adds/subtracts current amplitude from the selected electrodes, the electrode elements 704 are updated to reflect the changes. As the user changes the amplitude of current provided at the selected electrode the system calculates the new total current and the appropriate new fractionation of that current among the active electrodes. The GUI may include an “apply” element 710, whereby the new electrode configuration is calculated and transmitted to the IPG. According to some embodiments, the instructions/changes are transmitted to the IPG in terms of total current and how that current is divided amongst the electrodes, even though the user actually programmed those currents using current (i.e., mA) mode. In other words, they system executes algorithms (for example, remainder distribution) that calculates the appropriate total current and current fractionation to transmit to the IPG, which is the information the IPG expects to receive.

The illustrated GUI also comprises % Mode Controls 712 that can be used when the programming is performed in percentage (fractionation) mode. As explained above with respect to FIG. 5A, in percentage (fractionation) mode, the user may specify the total current to be applied and the percentage of that total to be applied at the various electrodes. According to some embodiments, the % Mode Controls may be locked out when the user has selected to use current (i.e., mA) mode, though the values of the total amplitude and the percentage applied to the selected electrode may be automatically updated as the user adjusts the current applied at each electrode.

According to some embodiments, the GUI may include further controls such as the deviation controls 714, which may be used to set and track a maximum amount that a given electrode can deviate from its prescribed current as the user adjusts other electrodes. For example, in the illustrated embodiment, the user has defined that the current assigned to electrode E3 should not deviate more than 0.01 mA as the user adjusts other electrodes. Presently, E3 has deviated-0.001 mA from the assigned value. The factors that may contribute to such deviations will be discussed below in more detail. The illustrated GUI also includes a reset element 716, which may be used to reset the currents (and/or fractionations) to a default value, for example, a value calculated based on a desired cathode pole or electric field geometry, as discussed above

As explained above, the system comprises one or more algorithms that are configured to accept an indication of an adjustment to one or more selected electrodes, wherein the adjustment comprises increasing or decreasing the amount of current to be delivered using the selected electrode(s). The algorithm calculates an amount by which the total current must be increased or decreased to accommodate the change. The algorithm then distributes the change in total current amongst the active electrodes to affect the desired change to the selected electrode while minimizing the changes to the currents applied at the other electrodes. The algorithm further computes the new total current and the new percentage (fractionations) for each of the active electrodes so that information can be provided to the IPG.

The programming algorithm may be stored in, and executed by, any of the components of the system illustrated in FIG. 4, for example. One skilled in the art will appreciate that programming algorithm can comprise a portion of software 96 operable in the clinician programmer 70 or other external system (FIG. 4). The programming algorithm can be stored as instructions on a computer-readable medium, such as on a magnetic or optical disk, in solid state memory, etc., and may be so stored in the clinician programmer 70 or in any external system (see FIG. 4).

A further aspect of the disclosed systems and algorithms involves monitoring and minimizing how the current on the current on the various electrodes may deviate from the currents assigned to those electrodes, especially as the currents are adjusted on the selected electrodes, as described above. Such deviation was alluded to above in the mention of the deviation controls 714 of the GUI 700.

Current deviations can occur because of limitations in the resolution of the IPG's stimulation circuitry, i.e., its DAC circuitry 28 (FIG. 3). Embodiments of DAC circuitry are described in detail in U.S. Pat. No. 10,912,942 (“the '942 Patent”), the contents of which are incorporated herein by reference. The architecture of the DAC circuitry will not be discussed here in detail, but the reader should appreciate some aspects of how the DAC circuitry produces the desired currents at the selected electrodes. As mentioned above, the DAC circuitry is activated and digitally programmed to produce the desired current with the correct timing (e.g., in accordance with the prescribed frequency F and pulse width PW). At a high level, the DAC architecture described in the '942 Patent achieves this in the following way: A master DAC (MDAC) receives an indication of a total anodic and total cathodic current amplitude “A” of the stimulation pulses that the IPG will form at any given time. As the charges should balance, the anodic A and cathodic A should be the same. In response, the MDAC sends a signal indicating the total anodic and cathodic current to the DACs for the individual electrodes (i.e., the DACs show in FIG. 3). That signal can comprise 8 bits in this described example, and thus, the MDAC can output currents in 256 increments. This impacts the resolution of the currents that the DACs can provide. If the total current window is 25.6 mA, then the highest resolution is +0.1 mA. If the total current is 2.56 mA, then the highest resolution is +0.01 mA.

In addition to the total current from the MDAC, each of the DACs also receive a fractionation control signal that indicates the percentage of the total that the DAC should provide. In the embodiments described in the '942 Patent, that fractionation control signal is variable in 1% increments (or lower in a low resolution mode). For example, a given electrode may be programmed to deliver 33% or 34% of the total current, but it cannot be programmed to deliver 33.333% of the total current.

The resolution limitations discussed here are related to DAC circuitry architectures, such as the ones described in the '942 Patent. Other architectures may present other resolution issues. But any DAC circuitry will inherently be limited in some way as to the resolution of current (or voltages) that may be applied at each of the electrodes.

These resolution limitations is one of the reasons that current on one or more of the electrodes may deviate from its assigned current as the currents on other electrodes are adjusted. For example, refer again to the example described in FIG. 6. In the initial state, a total cathodic current of 2 mA is divided evenly between E1 (1 mA) and E2 (1 mA). Recall that the user wants to add another 1.0 mA to electrode E2. In an unconstrained system, the system could simply add 1.0 mA to E2 (bringing its total to 2.0 mA) and leave E1 unchanged (i.e., with 1.0 mA on E1), thereby having a total 3.00 mA. In terms of percentages, that would provide 33.33 . . . % current on E1 and 66.66 . . . % current on E2. But recall that the resolution of the DAC circuitry is only capable of fractionating the current in 1% increments. So to best implement the user's input within the resolution limitations of the system, the algorithm changes the current on E1 to 0.99 mA and adds 2.01 mA (instead of the desired 2.00 mA) to E2.

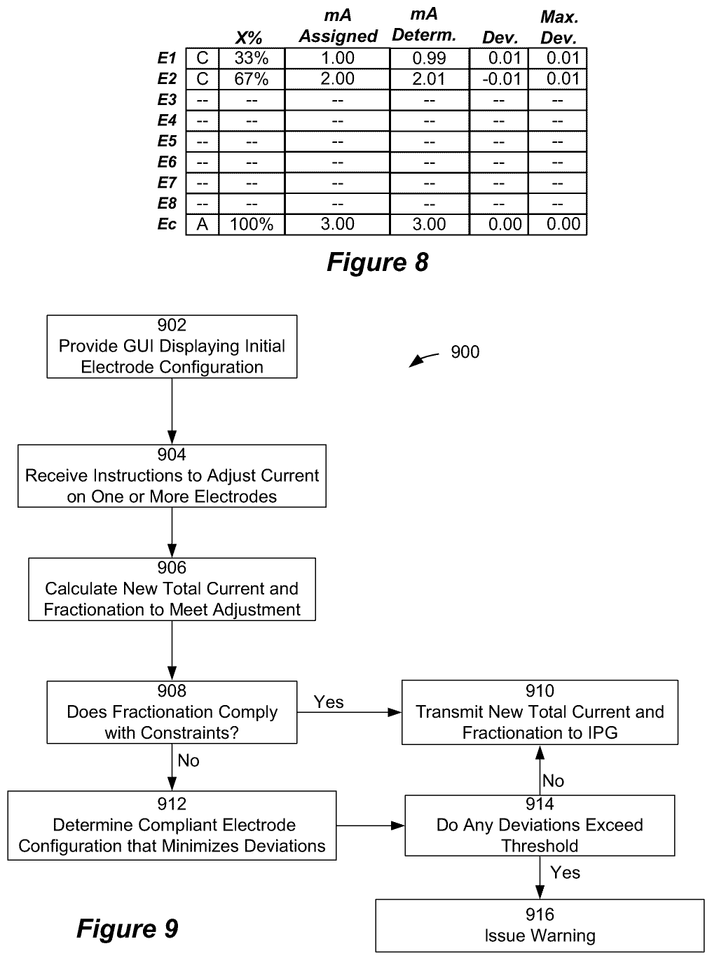

Notice that the new currents that the algorithm calculated for E1 and E2 in FIG. 6 deviate slightly from the currents that the user attempted to assign. According to some embodiments, the user may select a threshold defining the maximum deviation between the calculated and assigned currents. Referring to FIG. 7, the user can set a maximum (threshold) deviation for an electrode contact using the deviation controls 714. The deviation controls may also display an indication of the deviation for the selected electrode. According to some embodiments, as shown in FIG. 8, the algorithm may track the deviation between the current assigned by the user and the current determined by the algorithm. If the deviation in the current for a given exceeds the threshold specified by the user, the algorithm may issue a warning or otherwise indicate that the desired change to the current cannot be executed within the specified values.

FIG. 9 illustrates an embodiment of an algorithm 900 for adjusting current on a selected one or more electrodes using a current (i.e., mA) basis, as described herein. As an initial state assume an initial electrode configuration. At Step 902, a GUI may be displayed that reflects the initial electrode configuration. For example, the GUI such as the GUI 700 (FIG. 7) may displayed, which shows the present electrode configuration based on percentages of a total current on each electrode and/or the mA of current assigned to each electrode. At Step 904, the user chooses to adjust the current (using mA mode) on a selected electrode without changing the currents assigned to the other electrodes, as described above. According to some embodiments, the GUI may provide the option to adjust the electrode configuration using either fractionation (percentage) mode or current (mA) mode. In this example, assume that the user chooses to use current (mA) mode.

At Step 906, the algorithm determines the new total current that must be delivered by the electrode lead and how that current must be fractionated to comply with the user's request. In other words, the algorithm determines the new total current and the percentage of that total to be assigned to each electrode. At Step 908, the algorithm determines if the desired adjustment can be made within the constraints discussed above. For example, the algorithm determines if the new total current and the percentages at each electrode are within the resolution of the DAC circuitry. If the desired adjustment can be achieved within the constraints, then the algorithm transmits the new total current and fractionation information to the IPG (Step 910).

Step 912 and those that follow are implicated if the fractionation values initially calculated at Step 906 are not within the constraints of the system, for example, if they are not within the resolution limitations of the DAC circuitry. For example, if the calculate fractionation values require fractions of percentages at given electrodes but the DAC circuitry is only capable of providing 1% increments, then the calculated fractionation values do not comply with the constraints of the system. In that case, at Step 912, the algorithm mathematically calculates a new total current and a new fractionation of that total current between the electrodes that is within the constraints of the system (i.e., within the resolution capabilities of the DAC circuitry) and that minimizes the deviation between the determined electrode configuration and user's prescribed electrode configuration. The algorithm may use the mathematics of remainder distribution to minimize the differences between the calculated and assigned current values at each electrode. At Step 914 the algorithm may compare any deviations to a threshold determined by the user, as explained above. If the deviations do not exceed the threshold, then the algorithm transmits the calculated total current and fractionation values to the IPG (as per Step 910). If any of the current deviations exceed the threshold value, then the algorithm may issue a warning indicating as such (Step 916).

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A system for programming an implantable pulse generator (IPG), wherein the IPG is configurable to connect to a plurality of electrodes, the system comprising:
control circuitry configured to:
provide a graphical user interface (GUI) displaying an indication of an initial total current to be delivered by the plurality of electrodes and, for each of the electrodes, an initial percentage value indicating an initial percentage of the initial total current assigned to that electrode, wherein each initial percentage value defines an initial amplitude of current assigned to that electrode;
receive, via the GUI, an instruction to adjust the initial amplitude assigned to a selected one of the electrodes to an adjusted amplitude while maintaining the initial amplitudes for each of the non-selected electrodes;
calculate an adjusted total current and adjusted percentage values for each of the plurality of electrodes in accordance with the instruction, and
use the adjusted total current and the adjusted percentage values to program the IPG.

2. The system of claim 1, wherein the initial percentage values are configured to provide stimulation at a virtual pole at a position that is not a position of any of the electrodes.

3. The system of claim 1, wherein the GUI displays graphical representations of each of the plurality of electrodes such that each of the graphical representations are selectable by a user.

4. The system of claim 3, wherein the GUI displays GUI elements for adding and/or subtracting amplitude of current assigned to the selected electrode.

5. The system of claim 1, wherein calculating an adjusted total current adding the initial amplitudes of the non-selected electrodes and the adjusted amplitude of the selected electrode.

6. The system of claim 5, wherein calculating the adjusted percentage values for each of the plurality of electrodes comprises:
calculating an adjusted percentage value for the selected electrode by dividing the adjusted current for the selected electrode by the adjusted total current, and
calculating adjusted percentage values for each of the non-selected electrodes by dividing the initial amplitude for that that non-selected electrode by the adjusted total current.

7. The system of claim 1, wherein using the adjusted total current and the adjusted percentage values to program the IPG comprises:
determining if the IPG is capable of producing the adjusted total current and the adjusted percentage values,
if the IPG is capable of producing the adjusted total current and the adjusted percentage values, transmitting the adjusted total current and the adjusted percentage values to the IPG, and
if the IPG is not capable of producing the adjusted total current and the adjusted percentage values, calculating a modified adjusted total current and modified adjusted percentage values.

8. The system of claim 7, wherein determining if the IPG is capable of producing the adjusted total current and the adjusted percentage values comprises determining if the adjusted total current and the adjusted percentage values are within resolution capabilities of digital-to-analog (DAC) circuitry of the IPG.

9. The system of claim 7, wherein the modified adjusted total current and modified adjusted percentage values are calculated to minimize a deviation between the initial amplitude of current assigned to at least one of the non-elected electrodes and an amplitude of current defined for that non-elected electrode by the modified adjusted total current and modified adjusted percentage values.

10. The system of claim 9, wherein the GUI is configured to display an indication of the deviation between the initial amplitude of current assigned to the at least one of the non-elected electrodes and the amplitude of current defined for that non-elected electrode by the modified adjusted total current and modified adjusted percentage values.

11. The system of claim 9, wherein the control circuitry is configured to compare the deviation to a predetermined threshold value for the deviation and issue a warning if the deviation exceeds the threshold value.

12. The system of claim 11, wherein the GUI is configured to allow a user to set the predetermined threshold value for any of the plurality of electrodes.

13. The system of claim 1, wherein the GUI is configured to display the amplitude of current assigned to any of the plurality of electrodes.

14. A system for programming an implantable pulse generator (IPG), wherein the IPG is configurable to connect to a plurality of electrodes, the system comprising:
control circuitry configured to:
receive an indication of an amplitude of current to be assigned to a selected electrode,
use the amplitude of current to be assigned to the selected electrode to calculate a total current to be delivered by the plurality of electrodes and, for each of the electrodes, a percentage value indicating a percentage of the total current assigned to that electrode, and
transmit an indication of the total current and the percentage values to the IPG.

15. The system of claim 14, wherein calculating the total current comprises adding the amplitude of current assigned to the selected electrode to amplitudes of currents assigned to non-selected electrodes.

16. The system of claim 15, wherein calculating the percentage values for each of the plurality of electrodes comprises:
calculating a percentage value for the selected electrode by dividing the current for the selected electrode by the adjusted total current, and
calculating percentage values for each of the non-selected electrodes by dividing the amplitude for that that non-selected electrode by the total current.

17. The system of claim 14, wherein the control circuitry is further configured to use the total current and the percentage values to program the IPG.

18. The system of claim 17, wherein using the total current and the percentage values to program the IPG comprises:
determining if the IPG is capable of producing the total current and the percentage values,
if the IPG is capable of producing the total current and the percentage values, transmitting the total current and the percentage values to the IPG, and
if the IPG is not capable of producing the total current and the percentage values, calculating a modified total current and modified percentage values.

19. The system of claim 18, wherein determining if the IPG is capable of producing the total current and the percentage values comprises determining if the total current and the percentage values are within resolution capabilities of digital-to-analog (DAC) circuitry of the IPG.

20. The system of claim 18, wherein the modified total current and modified percentage values are calculated to minimize a deviation between the initial amplitude of current assigned to at least one of the non-elected electrodes and an amplitude of current defined for that non-elected electrode by the modified total current and modified percentage values.
